(19) United States
(12) Patent Application Publication
(10) Pub. No.: US 2024/0426674 A1
(43) Pub. Date: Dec. 26, 2024

(54) METHOD OF DETERMINING FOCUSING PARAMETER OF ELECTROMAGNETIC WAVES IN ELECTROMAGNETIC WAVE ENERGY TREATMENT SYSTEM AND ELECTROMAGNETIC WAVE ENERGY TREATMENT SYSTEM

(71) Applicant: ELECTRONICS AND TELECOMMUNICATIONS RESEARCH INSTITUTE, Daejeon (KR)

(72) Inventors: Wonyoung SONG, Sejong-si (KR); Kwang Jae LEE, Daejeon (KR); Soon Ik JEON, Daejeon (KR)

(73) Assignee: ELECTRONICS AND TELECOMMUNICATIONS RESEARCH INSTITUTE, Daejeon (KR)

(21) Appl. No.: 18/676,984

(22) Filed: May 29, 2024

(30) Foreign Application Priority Data
Jun. 20, 2023 (KR) 10-2023-0079156

(51) Int. Cl. G01K 3/00; A61N 5/10

(52) U.S. Cl. CPC G01K 3/005; A61N 5/1065

(57) ABSTRACT

A method of determining a focusing parameter of electromagnetic waves in an electromagnetic wave energy treatment system and an electromagnetic wave energy treatment system are provided. The method includes determining an initial focusing parameter based on a tomographic image of an object, predicting a temperature change of the object, when assuming that electromagnetic waves are irradiated to the object according to the initial focusing parameter, determining a final focusing parameter by optimizing a focusing parameter according to a prediction result of the temperature change, and irradiating electromagnetic waves to the object according to the final focusing parameter, wherein the prediction result of the temperature change may indicate whether an unnecessary focusing point has occurred in the object.

### CROSS-REFERENCE TO RELATED APPLICATION

This application claims the benefit of Korean Patent Application No. 10-2023-0079156, filed on Jun. 20, 2023, in the Korean Intellectual Property Office, the entire disclosure of which is incorporated herein by reference for all purposes.

### BACKGROUND

### 1. Field of the Invention

One or more embodiments relate to a method and system for determining a focusing parameter of electromagnetic waves in an electromagnetic wave energy treatment system.

### 2. Description of the Related Art

Recently, non-invasive treatment technology for treating lesions by irradiating high-density energy from outside the body to lesions inside the body has been in the spotlight as an alternative to invasive treatment technology that causes side effects, such as pain, physical burden, and aftereffects, to patients.

Non-invasive treatment includes radiation, ultrasonic waves, and electromagnetic waves, depending on the type of energy delivered. However, in the case of radiation, there is an issue of radiation exposure, and in the case of ultrasonic waves, there is an issue of being limited by bone and air structures. Accordingly, non-invasive treatment using electromagnetic waves is being studied.

An electromagnetic wave treatment system used in non-invasive treatment is technology for treating lesions by irradiating electromagnetic waves, in which the magnitude and phase are adjusted, from outside the body of a patient, focusing electromagnetic wave energy on the lesions, and increasing the temperature of the lesions by the energy.

However, in an existing electromagnetic wave treatment system, in a process of irradiating electromagnetic waves, there are cases that electromagnetic wave energy is focused in areas other than lesions and heat is generated. In this case, there is a possibility that the temperature of corresponding tissue may increase above normal temperature and side effects may occur.

Therefore, there is a demand for an electromagnetic wave energy treatment system that may prevent electromagnetic waves from being focused in areas other than target lesion areas.

### SUMMARY

Embodiments may provide a method and system for predicting or monitoring a temperature change in an electromagnetic wave energy treatment system to confirm whether an unnecessary focusing point occurs and when an unnecessary focusing point has occurred, readjusting a focusing parameter to prevent the unnecessary focusing point from occurring.

According to an aspect, there is provided a method of determining a focusing parameter of electromagnetic waves, the method including determining an initial focusing parameter based on a tomographic image of an object, predicting a temperature change of the object, when assuming that electromagnetic waves are irradiated to the object according to the initial focusing parameter, determining a final focusing parameter by optimizing a focusing parameter according to a prediction result of the temperature change, and irradiating electromagnetic waves to the object according to the final focusing parameter, wherein the prediction result of the temperature change may indicate whether an unnecessary focusing point has occurred in the object.

The determining of the initial focusing parameter may include converting the tomographic image into a numerical model including electromagnetic characteristics of a medium of the object, determining an analysis result by performing an electromagnetic analysis on the numerical model, and determining the initial focusing parameter based on the analysis result.

The predicting of the temperature change may include, when electromagnetic waves are irradiated to the object according to the initial focusing parameter, defining a focusing degree of electromagnetic waves as power loss density by predicting the focusing degree of electromagnetic waves for each area inside the object, predicting the temperature change in each area inside the object, based on a temperature increase range for each area inside the object and the power loss density, and confirming whether the unnecessary focusing point, which is an unintended energy focusing point of electromagnetic waves, has occurred by using a focusing target point corresponding to the temperature change in each area and the initial focusing parameter.

The determining of the final focusing parameter may include, when the unnecessary focusing point is confirmed not to have occurred in the prediction result of the temperature change, determining the initial focusing parameter as the final focusing parameter.

The determining of the final focusing parameter may include, when the unnecessary focusing point is confirmed to have occurred in the prediction result of the temperature change, extracting the unnecessary focusing point, determining an unnecessary focusing point removal parameter for removing the unnecessary focusing point, and determining a new final focusing parameter at which the unnecessary focusing point does not occur, by using the initial focusing parameter and the unnecessary focusing point removal parameter.

The unnecessary focusing point removal parameter may be a parameter corresponding to electromagnetic waves for generating, at the unnecessary focusing point, an electric field that has a same magnitude and an opposite phase with respect to an electric field generated at the unnecessary focusing point by the electromagnetic waves irradiated to the object according to the initial focusing parameter.

The determining of the final focusing parameter may further include, when electromagnetic waves are irradiated to the object according to the final focusing parameter, defining a focusing degree of electromagnetic waves as optimal power loss density by predicting the focusing degree of electromagnetic waves for each area inside the object, predicting the temperature change in each area inside the object, based on a temperature increase range for each area inside the object and the optimal power loss density, and confirming whether the unnecessary focusing point has occurred by using a focusing target point corresponding to the temperature change in each area and the final focusing parameter, wherein the determining of the final focusing parameter may be repeatedly performed until the unnecessary focusing point does not occur.

The method may further include monitoring temperature of the object to which electromagnetic waves are irradiated according to the final focusing parameter and readjusting the final focusing parameter based on a monitoring result.

The readjusting of the final focusing parameter may include confirming, based on the monitoring result, whether an unnecessary temperature increase has occurred, in which temperature increases above a threshold value in an area other than a focusing target point inside the object, when the unnecessary temperature increase has occurred, extracting an area in which the unnecessary temperature increase has occurred as a new unnecessary focusing point, when a position of the unnecessary focusing point is identical to a position of the new unnecessary focusing point, readjusting the final focusing parameter, and irradiating electromagnetic waves to the object according to the readjusted final focusing parameter.

The readjusting of the final focusing parameter may further include, when the unnecessary focusing point is different from the new unnecessary focusing point, determining a new unnecessary focusing point removal parameter for removing the new unnecessary focusing point, determining a new final focusing parameter at which the unnecessary focusing point does not occur, by using the final focusing parameter and the new unnecessary focusing point removal parameter, and irradiating electromagnetic waves to the object according to the new final focusing parameter.

According to another aspect, there is provided an electromagnetic wave energy treatment system including a parameter operator configured to determine an initial focusing parameter based on a tomographic image of an object, predict a temperature change of the object, when assuming that electromagnetic waves are irradiated to the object according to the initial focusing parameter, and determine a final focusing parameter by optimizing a focusing parameter according to a prediction result of the temperature change, and an electromagnetic wave irradiator configured to irradiate electromagnetic waves to the object according to the final focusing parameter, wherein the prediction result of the temperature change may indicate whether an unnecessary focusing point has occurred in the object.

The parameter operator may be further configured to convert the tomographic image into a numerical model including electromagnetic characteristics of a medium of the object, determine an analysis result by performing an electromagnetic analysis on the numerical model, and determine the initial focusing parameter based on the analysis result.

The parameter operator may be further configured to, when electromagnetic waves are irradiated to the object according to the initial focusing parameter, define a focusing degree of electromagnetic waves as power loss density by predicting the focusing degree of electromagnetic waves for each area inside the object, predict the temperature change in each area inside the object, based on a temperature increase range for each area inside the object and the power loss density, and confirm whether the unnecessary focusing point, which is an unintended energy focusing point of electromagnetic waves, has occurred by using a focusing target point corresponding to the temperature change in each area and the initial focusing parameter.

The parameter operator may be further configured to, when the unnecessary focusing point is confirmed not to have occurred in the prediction result of the temperature change, determine the initial focusing parameter as the final focusing parameter.

The parameter operator may be further configured to, when the unnecessary focusing point is confirmed to have occurred in the prediction result of the temperature change, extract the unnecessary focusing point, determine an unnecessary focusing point removal parameter for removing the unnecessary focusing point, and determine a new final focusing parameter at which the unnecessary focusing point does not occur, by using the initial focusing parameter and the unnecessary focusing point removal parameter.

The unnecessary focusing point removal parameter may be a parameter corresponding to electromagnetic waves for generating, at the unnecessary focusing point, an electric field that has a same magnitude and an opposite phase with respect to an electric field generated at the unnecessary focusing point by the electromagnetic waves irradiated to the object according to the initial focusing parameter.

The parameter operator may be further configured to, when electromagnetic waves are irradiated to the object according to the final focusing parameter, define a focusing degree of electromagnetic waves as optimal power loss density by predicting the focusing degree of electromagnetic waves for each area inside the object, predict the temperature change in each area inside the object, based on a temperature increase range for each area inside the object and the optimal power loss density, and confirm whether the unnecessary focusing point has occurred by using a focusing target point corresponding to the temperature change in each area and the final focusing parameter.

The electromagnetic wave energy treatment system may further include a temperature monitor configured to monitor temperature of the object to which electromagnetic waves are irradiated according to the final focusing parameter, wherein the parameter operator may be further configured to readjust the final focusing parameter based on a monitoring result.

The parameter operator may be further configured to confirm, based on the monitoring result, whether an unnecessary temperature increase has occurred, in which temperature increases above a threshold value in an area other than a focusing target point inside the object, when the unnecessary temperature increase has occurred, extract an area in which the unnecessary temperature increase has occurred as a new unnecessary focusing point, and when a position of the unnecessary focusing point is identical to a position of the new unnecessary focusing point, readjust the final focusing parameter, wherein the electromagnetic wave irradiator may be further configured to irradiate electromagnetic waves to the object according to the readjusted final focusing parameter.

The parameter operator may be further configured to, when the unnecessary focusing point is different from the new unnecessary focusing point, determine a new unnecessary focusing point removal parameter for removing the new unnecessary focusing point, determine a new final focusing parameter at which the unnecessary focusing point does not occur, by using the final focusing parameter and the new unnecessary focusing point removal parameter, wherein the electromagnetic wave irradiator may be further configured to irradiate electromagnetic waves to the object according to the new final focusing parameter.

Additional aspects of embodiments will be set forth in part in the description which follows and, in part, will be apparent from the description, or may be learned by practice of the disclosure.

According to embodiments, a temperature change in an electromagnetic wave energy treatment system may be predicted or monitored to confirm whether an unnecessary focusing point occurs and when an unnecessary focusing point has occurred, a focusing parameter may be readjusted to prevent the unnecessary focusing point from occurring. Accordingly, leakage of electromagnetic wave energy may be prevented and electromagnetic wave energy may concentrate only at a focusing target point so that the irradiation efficiency of electromagnetic waves and treatment efficiency due to irradiation of electromagnetic waves may be increased.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a diagram illustrating an electromagnetic wave energy treatment system including a function of determining a focusing parameter of electromagnetic waves, according to an embodiment;

FIG. 2 illustrates an example of a process of removing an unnecessary focusing point, according to an embodiment;

FIG. 3 illustrates an example of a result of removing an unnecessary focusing point, according to an embodiment;

FIG. 4 illustrates an example of a result of irradiating electromagnetic waves using a focusing parameter, according to an embodiment;

FIG. 5 is a flowchart illustrating a method of determining a focusing parameter of electromagnetic waves, according to an embodiment;

FIG. 6 is a flowchart illustrating a focusing parameter optimization process of a method of determining a focusing parameter of electromagnetic waves, according to an embodiment; and

FIG. 7 is a flowchart illustrating a focusing parameter readjustment process of a method of determining a focusing parameter of electromagnetic waves, according to an embodiment.

### DETAILED DESCRIPTION

Hereinafter, embodiments will be described in detail with reference to the accompanying drawings. A method of determining a focusing parameter of electromagnetic waves, according to an embodiment of the present disclosure, may be performed by a system for determining a focusing parameter of electromagnetic waves.

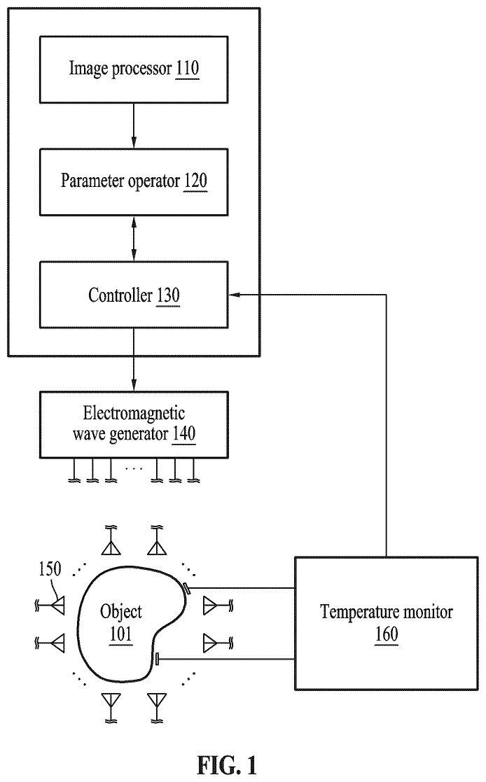

FIG. 1 is a diagram illustrating an electromagnetic wave energy treatment system including a function of determining a focusing parameter of electromagnetic waves, according to an embodiment.

The electromagnetic wave energy treatment system may irradiate electromagnetic waves to an object 101 and may deliver electromagnetic wave energy to a focusing target point inside the object 101, thereby increasing the temperature of the focusing target point and treating lesions at the focusing target point. Here, the focusing target point may be a point where a lesion has occurred on the body of a patient or an area within a certain distance from the point where the lesion has occurred.

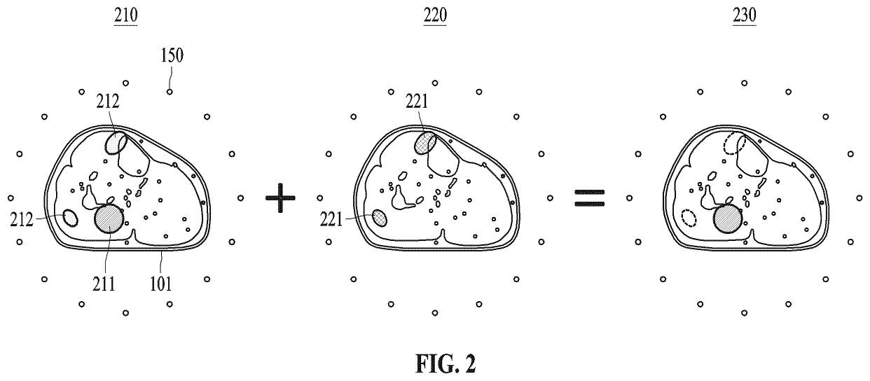

As shown in FIG. 1, the electromagnetic wave energy treatment system including a function of determining a focusing parameter of electromagnetic waves may include an image processor 110, a parameter operator 120, a controller 130, an electromagnetic wave generator 140, an electromagnetic wave irradiator 150, and a temperature monitor 160. Here, the image processor 110, the parameter operator 120, and the controller 130 may be different processors, as shown in FIG. 2, and may each be a module included in a program stored in a storage medium and executed by one processor. In addition, the object 101 may be a part of the body of a patient to which electromagnetic waves are to be irradiated for treatment.

The image processor 110 may receive or load a tomography image of the object 101. For example, the tomography image may be an internal tomography image of the object 101 included in a medical image of a patient. In addition, the image processor 110 may convert the tomography image into a numerical model including the electromagnetic characteristics of a medium of the object 101. The numerical model may be a model required for electromagnetic numerical analysis and may include information such as relative permittivity and conductivity of each area of the object 101. Furthermore, the image processor 110 may classify what each area of the object 101 is composed of in the tomography image of the object 101. The image processor 110 may generate the numerical model by mapping the relative permittivity and conductivity corresponding to each area of the object 101 to each area of the object 101, according to a classification result.

The parameter operator 120 may determine an analysis result by performing electromagnetic analysis on the numerical model from conversion by the image processor 110. For example, the parameter operator 120 may obtain an electromagnetic analysis result by inputting the numerical model into an electromagnetic analysis process. The electromagnetic analysis process may be a process of analyzing how an electric field is formed inside the object 101 when electromagnetic waves are emitted from an antenna located outside the object 101. For example, the electromagnetic analysis result may be a Green's function inside the object 101 for each antenna. The Green's function may be a solution to a differential equation if a source is a delta function ( ) In addition, the delta function ( ) may be a function that has a value only when x included in ( ) is 0.

Furthermore, the parameter operator 120 may determine an initial focusing parameter based on the analysis result. For example, the parameter operator 120 may obtain the initial focusing parameter by inputting the analysis result into a focusing parameter operation process.

Here, assuming that electromagnetic waves are irradiated to the object 101 according to the initial focusing parameter, the parameter operator 120 may predict a temperature change of the object 101 according to the assumption. In addition, the parameter operator 120 may determine a final focusing parameter by optimizing a focusing parameter according to a prediction result of the temperature change. Here, the prediction result of the temperature change may be information indicating whether an unnecessary focusing point has occurred in the object 101. The unnecessary focusing point may be an energy focusing point of electromagnetic waves, which is unintended by a user. Specifically, the unnecessary focusing point may be a point that is not a focusing target point and the difference with the temperature of the focusing target point is less than or equal to a threshold value.

Specifically, when electromagnetic waves are irradiated to the object 101 according to the initial focusing parameter, the parameter operator 120 may predict a focusing degree of electromagnetic waves for each area inside the object 101 and may define the focusing degree of electromagnetic waves as power loss density. Subsequently, the parameter operator 120 may predict the temperature change in each area inside the object 101, based on a temperature increase range for each area inside the object 101 and power loss density. Here, the temperature increase range for each area inside the object 101 may be information indicating the temperature change according to a change in electromagnetic waves for each area within the body of a patient and may vary depending on factors such as metabolism and blood flow of tissues of the body of the patient. Subsequently, the parameter operator 120 may confirm whether the unnecessary focusing point has occurred by using the temperature change in each area and the focusing target point corresponding to the initial focusing parameter. For example, the parameter operator 120 may predict the temperature change in each area inside the object 101 using a bio-heat equation.

However, when the same electromagnetic waves are continuously irradiated to the object 101, the temperature of the object 101 may increase rapidly, but the rate of increase may slow down over time and may reach saturation at a certain temperature. Therefore, the temperature change in each area of the object 101 over the total time may be nonlinear. However, if the bio-heat equation including partial differential equations is applied to a nonlinear operation, an operational load may occur.

In addition, when the total time is divided into a predetermined time interval, the temperature change in each area of the object 101 may be linear in each time interval. Accordingly, the parameter operator 120 may divide the total time, from the start of irradiating electromagnetic waves to the object 101 to the end of irradiating the electromagnetic waves, into the predetermined time interval to generate time sections. Subsequently, the parameter operator 120 may apply the bio-heat equation to each of the time sections so that the operational load may be minimized and the temperature change in each area inside the object 101 may be predicted.

When the unnecessary focusing point is confirmed not to have occurred in the prediction result of the temperature change, the parameter operator 120 may determine the initial focusing parameter as the final focusing parameter.

When the unnecessary focusing point is confirmed to have occurred in the prediction result of the temperature change, the parameter operator 120 may extract the unnecessary focusing point. Subsequently, the parameter operator 120 may determine an unnecessary focusing point removal parameter for removing the unnecessary focusing point. Here, the unnecessary focusing point removal parameter may be a parameter corresponding to electromagnetic waves for generating, at the unnecessary focusing point, an electric field that has the same magnitude and an opposite phase with respect to an electric field generated at the unnecessary focusing point by the electromagnetic waves irradiated to the object 101 according to the initial focusing parameter. In addition, the parameter operator 120 may determine the final focusing parameter at which the unnecessary focusing point does not occur, by using the initial focusing parameter and the unnecessary focusing point removal parameter.

Furthermore, when electromagnetic waves are irradiated to the object 101 according to the final focusing parameter, the parameter operator 120 may predict the focusing degree of electromagnetic waves for each area inside the object 101 and may define the focusing degree of electromagnetic waves as optimal power loss density. Subsequently, the parameter operator 120 may predict the temperature change in each area inside the object 101, based on the temperature increase range for each area inside the object 101 and the optimal power loss density. Next, the parameter operator 120 may confirm whether the unnecessary focusing point has occurred by using the temperature change in each area and the focusing target point corresponding to the final focusing parameter.

The parameter operator 120 may determine the final focusing parameter, which prevents the unnecessary focusing point from occurring, by repeatedly performing the above-described process until the unnecessary focusing point does not occur.

The controller 130 may control the electromagnetic wave generator 140 to generate electromagnetic waves having a magnitude and a phase corresponding to the final focusing parameter determined by the parameter operator 120. For example, the controller 130 may transmit a command to the electromagnetic wave generator 140 to generate electromagnetic waves having a magnitude and a phase corresponding to the final focusing parameter.

The electromagnetic wave generator 140 may generate electromagnetic waves having a magnitude and a phase corresponding to the final focusing parameter according to control by the controller 130. For example, the electromagnetic wave generator 140 may include an amplifier for adjusting the magnitude of the generated electromagnetic waves and a phase shifter for adjusting the phase of the generated electromagnetic waves. In addition, the electromagnetic waves generated by the electromagnetic wave generator 140 may be high-power electromagnetic waves of 100 watts (W) or more.

The electromagnetic wave irradiator 150 may radiate the electromagnetic waves generated by the electromagnetic wave generator 140 to the object 101. Here, the electromagnetic wave irradiator 150 may include an array of a plurality of irradiators, as shown in FIG. 1.

The temperature monitor 160 may monitor the temperature of the object 101 to which electromagnetic waves are irradiated by the electromagnetic wave irradiator 150. For example, the temperature monitor 160 may be a medical device such as a magnetic resonance imaging (MRI) device or may be a separate sensor or device for measuring the internal temperature of the object 101. In addition, the temperature monitor 160 may be a device for estimating a change in the internal temperature of the object 101 through simulation.

Here, the parameter operator 120 may readjust the final focusing parameter based on the monitoring result of the temperature monitor 160.

Specifically, based on the monitoring result, the parameter operator 120 may confirm whether an unnecessary temperature increase has occurred, in which temperature increases above a threshold value in an area other than the focusing target point inside the object 101.

When the unnecessary temperature increase has occurred, the parameter operator 120 may extract an area in which the unnecessary temperature increase has occurred as a new unnecessary focusing point. In addition, the parameter operator 120 may confirm whether the position of the unnecessary focusing point extracted before irradiating electromagnetic waves is identical to the position of the new unnecessary focusing point.

When the position of the unnecessary focusing point is identical to the position of the new unnecessary focusing point, the parameter operator 120 may readjust the final focusing parameter. Here, the electromagnetic wave generator 140 may generate electromagnetic waves corresponding to the final focusing parameter readjusted by the parameter operator 120. In addition, the electromagnetic wave irradiator 150 may irradiate electromagnetic waves generated by the electromagnetic wave generator 140 to the object 101.

When the unnecessary focusing point is different from the new unnecessary focusing point, the parameter operator 120 may determine a new unnecessary focusing point removal parameter for removing the new unnecessary focusing point. Subsequently, the parameter operator 120 may determine a new final focusing parameter at which the unnecessary focusing point does not occur, by using the final focusing parameter and the new unnecessary focusing point removal parameter. Here, the electromagnetic wave generator 140 may generate electromagnetic waves corresponding to the new final focusing parameter determined by the parameter operator 120. In addition, the electromagnetic wave irradiator 150 may irradiate electromagnetic waves generated by the electromagnetic wave generator 140 to the object 101.

Furthermore, according to an embodiment, as shown in FIG. 1, the temperature monitor 160 may monitor the temperature change of the object 101, but depending on embodiments, the temperature monitor 160 may monitor information such as electromagnetic waves inside the object 101 and may determine or adjust the final focusing parameter based on the monitoring result.

In the present disclosure, a temperature change in an electromagnetic wave energy treatment system may be predicted or monitored to confirm whether an unnecessary focusing point occurs and when an unnecessary focusing point has occurred, a focusing parameter may be readjusted to prevent the unnecessary focusing point from occurring. Accordingly, leakage of electromagnetic wave energy may be prevented and electromagnetic wave energy may concentrate only at a focusing target point so that the irradiation efficiency of electromagnetic waves and treatment efficiency due to irradiation of electromagnetic waves may be increased.

FIG. 2 illustrates an example of a process of removing an unnecessary focusing point, according to an embodiment.

An image 210 of FIG. 2 may be a result of predicting a temperature change of the object 101 when the electromagnetic wave irradiator 150 irradiates, to the object 101, electromagnetic waves according to an initial focusing parameter W_{f}. In addition, an image 220 of FIG. 2 may be a result of predicting the temperature change of the object 101 when the electromagnetic wave irradiator 150 irradiates, to the object 101, electromagnetic waves according to an unnecessary focusing point removal parameter Wₕ. Furthermore, an image 230 of FIG. 2 may be a result of predicting the temperature change of the object 101 when the electromagnetic wave irradiator 150 irradiates, to the object 101, electromagnetic waves according to a final focusing parameter Wₒₚₜ.

The electromagnetic waves according to the initial focusing parameter W_{f} may increase the temperature of a focusing target point 211 and the temperature of an unnecessary focusing point 212 above a threshold value, as shown in FIG. 2. In addition, the unnecessary focusing point removal parameter Wₕ may be a parameter for generating, at the unnecessary focusing point 212, electromagnetic waves that have the same magnitude and an opposite phase with respect to an electric field generated at the unnecessary focusing point 212 in the image 210. For example, the parameter operator 120 may determine the unnecessary focusing point removal parameter Wₕ using Equation 1.

$${w}_{h} = {\sum }_{p = 1}^{P} {a}_{p} {v}_{hp} = [ \begin{matrix}{v}_{h 1} & {v}_{h 2} & … & {v}_{hP}\end{matrix} ] {a}_{h} \qquad [ Equation 1 ]$$

Here, if the p-th unnecessary focusing point rₚ is set as a focusing target point, Vₕₚ may be a focusing parameter that allows electromagnetic waves to be focused on rₚ. In addition, αₚ may be a weighted value for the focusing parameter and may be unknown. As shown in Equation 2, αₕ may be a column vector in which a transpose function T is applied to αₚ.

$${a}_{h} = {[ {a}_{1} , {a}_{2} , … , {a}_{P} ]}^{T} \qquad [ Equation 2 ]$$

As shown in Equation 3, when the parameter operator 120 generates the final focusing parameter Wₒₚₜ by adding the initial focusing parameter W_{f} to the unnecessary focusing point removal parameter Wₕ, the electromagnetic wave irradiator 150 may irradiate electromagnetic waves in which the temperature increases only at the focusing target point 211 without increasing the temperature at the unnecessary focusing point 212, as shown in the image 230.

$${w}_{opt} = {w}_{f} + {w}_{h} \qquad [ Equation 3 ]$$

Specifically, when the initial focusing parameter W_{f} is used, the parameter operator 120 may determine E(rₚ) which is an electric field value at the generated unnecessary focusing point 212 rₚ. For example, the parameter operator 120 may determine E(rₚ) using Equation 4.

$$E ( {r}_{p} ) = {g}_{Tx} ( {r}_{p} ) {w}_{f} \qquad [ Equation 4 ]$$

Here, g_{Tx} may be a Green's function inside the object 101 output as an electromagnetic analysis result. The parameter operator 120 may define the Green's function g_{Tx} within the object 101 for one antenna. In addition, the parameter operator 120 may operate the Green's function g_{Tx}(rₚ), at the p-th unnecessary focusing point rₚ, and the initial focusing parameter W_{f} to determine E(rₚ).

Subsequently, the parameter operator 120 may determine the focusing parameter Vₕₚ, in which the unnecessary focusing point 212 rₚ is set as a focusing target point. In addition, when the focusing parameter Vₕₚ is used, the parameter operator 120 may determine E(rₚ), which is the electric field value at the generated unnecessary focusing point 212 rₚ. For example, the parameter operator 120 may determine E(rₚ) using Equation 5.

$${E ( {r}_{p} )}_{=} {g}_{Tx} ( {r}_{p} ) {v}_{hp} \qquad [ Equation 5 ]$$

As described above, the final focusing parameter Wₒₚₜ may be generated by adding the initial focusing parameter W_{f} to the unnecessary focusing point removal parameter Wₕ. Since the electric field follows the principle of superposition, when the final focusing parameter Wₒₚₜ is used, E (rₚ, which is the electric field value at the generated unnecessary focusing point 212 rₚ, may be as shown in Equation 6.

$${E ( {r}_{p} )}_{=} {g}_{Tx} ( {r}_{p} ) ( {w}_{f} + {w}_{h} ) \qquad [ Equation 6 ]$$

Here, the parameter operator 120 may determine the unnecessary focusing point removal parameter Wₕ by determining a value of αₚ that allows E (to become 0.

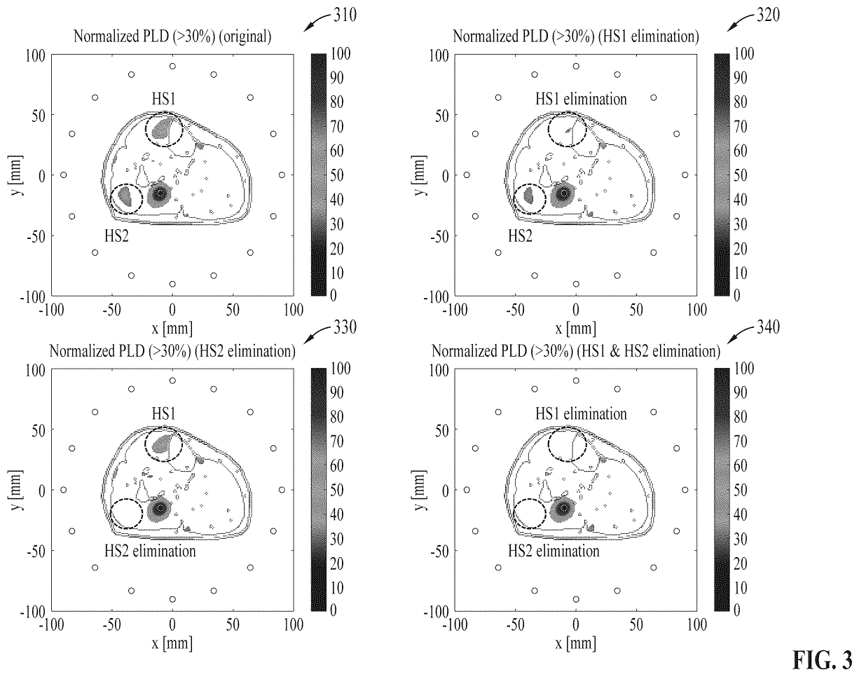

FIG. 3 illustrates an example of a result of removing an unnecessary focusing point, according to an embodiment.

According to an embodiment, the parameter operator 120 may determine the final focusing parameter Wₒₚₜ for removing only one of unnecessary focusing points by determining the focusing parameter Vₕₚ that has set the p-th unnecessary focusing point rₚ among the unnecessary focusing points as a focusing target point.

An image 310 of FIG. 3 may be a result of predicting a temperature change of the object 101 when the electromagnetic wave irradiator 150 irradiates, to the object 101, electromagnetic waves according to the initial focusing parameter W_{f}.

For example, the focusing parameter Vₕₚ may be determined in which the parameter operator 120 has set only an unnecessary focusing point HS1 as a focusing target point, the unnecessary focusing point removal parameter Wₕ may be determined using the focusing parameter Vₕₚ, and the final focusing parameter Wₒₚₜ may be determined using the unnecessary focusing point removal parameter Wₕ. Here, as a result of irradiating electromagnetic waves according to the final focusing parameter Wₒₚₜ to the object 101, only the unnecessary focusing point HS1 may be removed and an unnecessary focusing point HS2 may be maintained, as shown in an image 320 of FIG. 3.

In addition, the focusing parameter Vₕₚ may be determined in which the parameter operator 120 has set only the unnecessary focusing point HS2 as a focusing target point, the unnecessary focusing point removal parameter Wₕ may be determined using the focusing parameter Vₕₚ, and the final focusing parameter Wₒₚₜ may be determined using the unnecessary focusing point removal parameter Wₕ. Here, as a result of irradiating electromagnetic waves according to the final focusing parameter Wₒₚₜ to the object 101, only the unnecessary focusing point HS2 may be removed and the unnecessary focusing point HS1 may be maintained, as shown in an image 330 of FIG. 3.

Furthermore, the focusing parameter Vₕₚ may be determined in which the parameter operator 120 has set both the unnecessary focusing point HS1 and the unnecessary focusing point HS2 as focusing target points, the unnecessary focusing point removal parameter Wₕ may be determined using the focusing parameter Vₕₚ, and the final focusing parameter Wₒₚₜ may be determined using the unnecessary focusing point removal parameter Wₕ. Here, as a result of irradiating electromagnetic waves according to the final focusing parameter Wₒₚₜ to the object 101, both the unnecessary focusing point HS1 and the unnecessary focusing point HS2 may be removed, as shown in an image 340 of FIG. 3.

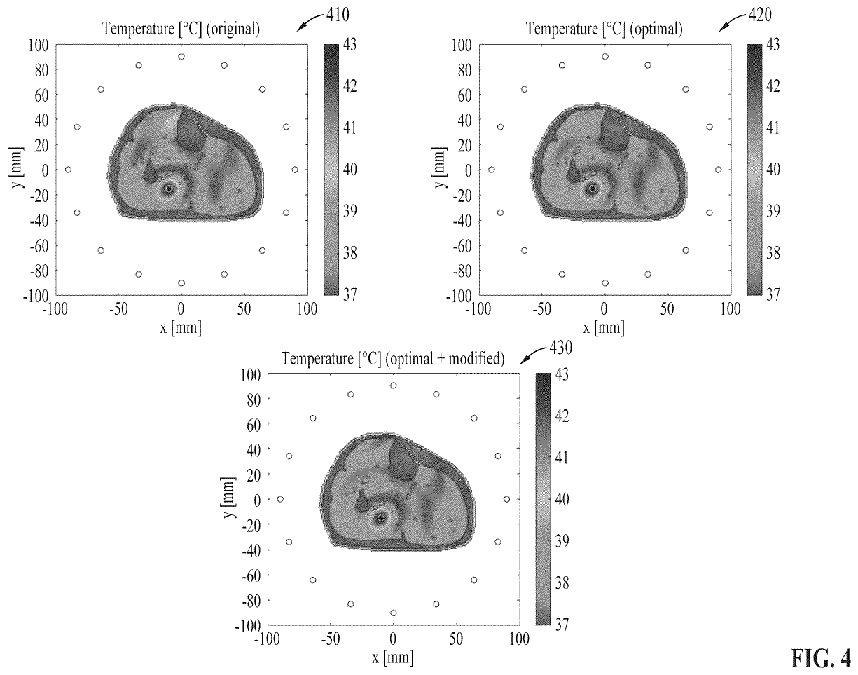

FIG. 4 illustrates an example of a result of irradiating electromagnetic waves using a focusing parameter, according to an embodiment.

An image 410 of FIG. 4 is an example of a result of measuring temperature of the object 101 when the electromagnetic wave irradiator 150 irradiates, to the object 101, electromagnetic waves according to an initial focusing parameter.

An image 420 of FIG. 4 is an example of a result of measuring temperature of the object 101 when the electromagnetic wave irradiator 150 irradiates, to the object 101, electromagnetic waves according to a final focusing parameter. As shown in the image 420, the temperature of an unnecessary focusing point located at the 12 o'clock position of the object 101 may be lower than that of the image 410, but the unnecessary focusing point may not be removed.

When the temperature monitor 160 measures the result as shown in the image 420 of FIG. 4, the parameter operator 120 may readjust the final focusing parameter or may determine a new final focusing parameter. In addition, an image 430 of FIG. 4 is an example of a result of measuring temperature of the object 101 when the electromagnetic wave irradiator 150 radiates, to the object 101, electromagnetic waves according to the readjusted final focusing parameter or the new final focusing parameter. As shown in the image 430, the unnecessary focusing point located at the 12 o'clock position may be removed.

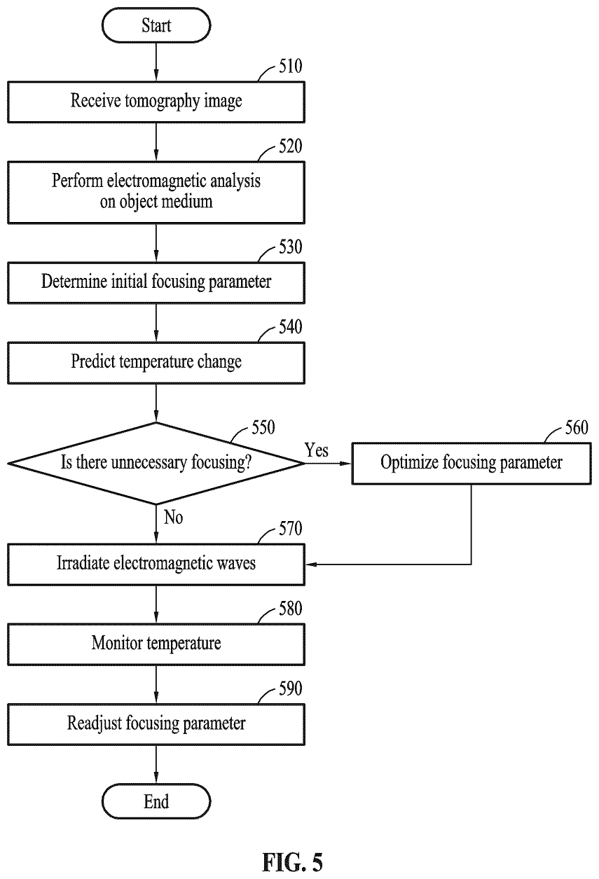

FIG. 5 is a flowchart illustrating a method of determining a focusing parameter of electromagnetic waves, according to an embodiment.

In operation 510, the image processor 110 may receive or load a tomography image of the object 101. For example, the tomography image may be an internal tomography image of the object 101 included in a medical image of a patient. In addition, the image processor 110 may convert the received or loaded tomographic image into a numerical model including the electromagnetic characteristics of a medium of the object 101.

In operation 520, the parameter operator 120 may determine an analysis result by performing electromagnetic analysis on the numerical model from the conversion in operation 510.

In operation 530, the parameter operator 120 may determine an initial focusing parameter based on the analysis result in operation 520.

In operation 530, when assuming that electromagnetic waves are irradiated to the object according to the initial focusing parameter, the parameter operator 120 may predict a temperature change of the object 101 according to the assumption.

In operation 540, the parameter operator 120 may confirm whether an unnecessary focusing point has occurred in the object 101. When the unnecessary focusing point is confirmed not to have occurred in a prediction result of the temperature change, the parameter operator 120 may determine the initial focusing parameter as the final focusing parameter and may perform operation 570. When the unnecessary focusing point is confirmed to have occurred in the prediction result of the temperature change, the parameter operator 120 may perform operation 560.

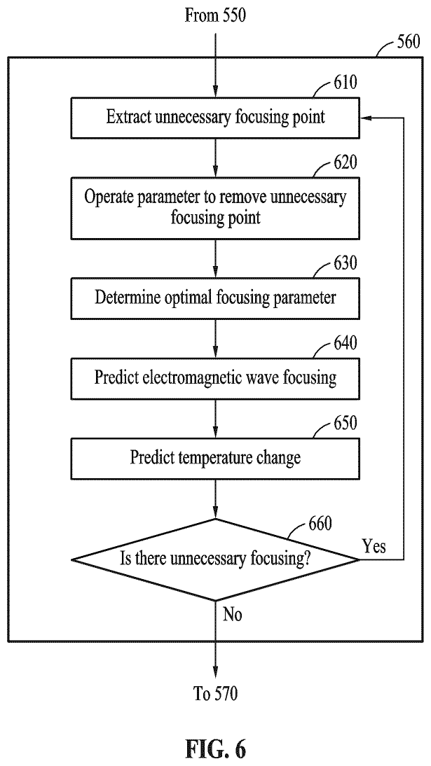

In operation 560, the parameter operator 120 may optimize the focusing parameter and may determine the final focusing parameter. A process of determining the final focusing parameter by the parameter operator 120 is described in detail below with reference to FIG. 6.

In operation 570, the electromagnetic wave generator 140 may generate electromagnetic waves of which the magnitude and the phase are determined according to the final focusing parameter determined by the parameter operator 120. In addition, the electromagnetic wave irradiator 150 may irradiate electromagnetic waves generated by the electromagnetic wave generator 140 to the object 101.

In operation 580, the temperature monitor 160 may monitor the temperature of the object 101 to which electromagnetic waves are irradiated by the electromagnetic wave irradiator 150.

In operation 590, the parameter operator 120 may readjust the final focusing parameter based on a monitoring result of the temperature monitor 160.

In operation 570, when the electromagnetic wave irradiator 150 actually irradiates electromagnetic waves to the object 101, the unnecessary focusing point may not be completely removed or a new unnecessary focusing point may occur, unlike a focusing prediction result, due to errors in the electromagnetic wave irradiator 150, hardware errors, etc. Accordingly, the parameter operator 120 may receive the monitoring result of the temperature monitor 160 and may readjust the final focusing parameter based on the monitoring result of the temperature monitor 160, thereby removing the unnecessary focusing point.

FIG. 6 is a flowchart illustrating a focusing parameter optimization process of a method of determining a focusing parameter of electromagnetic waves, according to an embodiment. Operations 610 to 660 of FIG. 6 may be included in operation 560 of FIG. 5.

In operation 610, the parameter operator 120 may extract an unnecessary focusing point.

In operation 620, the parameter operator 120 may determine an unnecessary focusing point removal parameter for removing the unnecessary focusing point. Here, the unnecessary focusing point removal parameter may be a parameter corresponding to electromagnetic waves for generating, at the unnecessary focusing point, an electric field that has the same magnitude and an opposite phase with respect to an electric field generated at the unnecessary focusing point by the electromagnetic waves irradiated to the object 101 according to an initial focusing parameter.

In operation 630, the parameter operator 120 may determine a final focusing parameter at which the unnecessary focusing point does not occur using the initial focusing parameter and the unnecessary focusing point removal parameter.

In operation 640, when electromagnetic waves are irradiated to the object 101 according to the final focusing parameter, the parameter operator 120 may define a focusing degree of electromagnetic waves as optimal power loss density by predicting the focusing degree of electromagnetic waves for each area inside the object 101.

In operation 650, the parameter operator 120 may predict a temperature change in each area inside the object 101, based on a temperature increase range for each area inside the object 101 and the optimal power loss density.

In operation 660, the parameter operator 120 may confirm whether the unnecessary focusing point has occurred by using a focusing target point corresponding to the temperature change in each area and the final focusing parameter. When the unnecessary focusing point has not occurred, the parameter operator 120 may terminate the operation. When the unnecessary focusing point has occurred, the parameter operator 120 may determine the final focusing parameter for preventing the unnecessary focusing point from occurring, by repeatedly performing operations 610 to 660 until the unnecessary focusing point does not occur.

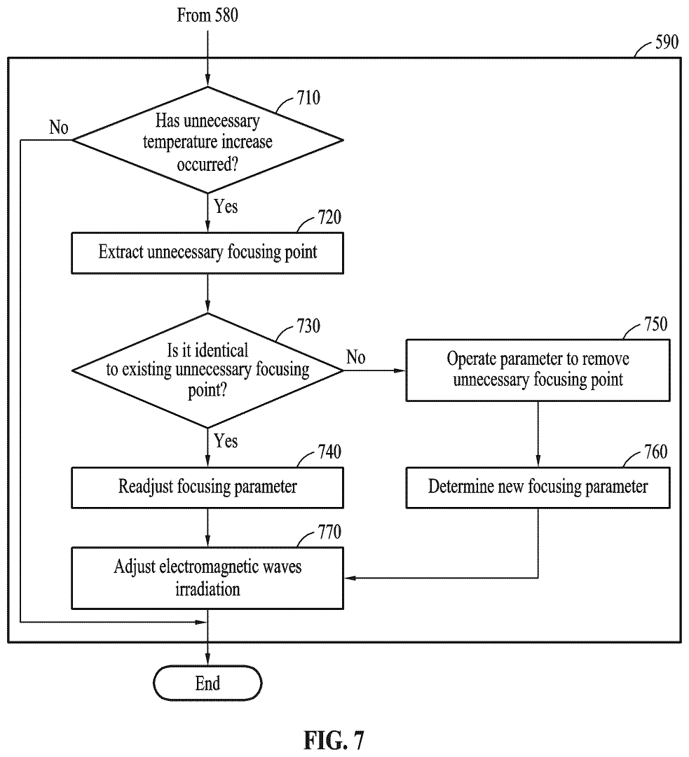

FIG. 7 is a flowchart illustrating a focusing parameter readjustment process of a method of determining a focusing parameter of electromagnetic waves, according to an embodiment. Operations 710 to 770 of FIG. 7 may be included in operation 590 of FIG. 5.

In operation 710, the parameter operator 120 may confirm, based on the monitoring result in operation 580, whether an unnecessary temperature increase has occurred, in which temperature increases above a threshold value in an area other than a focusing target point inside the object 101. When the unnecessary temperature increase has occurred, the parameter operator 120 may perform operation 720. In addition, when the unnecessary temperature increase has not occurred, the parameter operator 120 may determine that the unnecessary focusing point has been removed, like a focusing prediction result, and may terminate the operation.

In operation 720, the parameter operator 120 may extract an area in which the unnecessary temperature increase has occurred as a new unnecessary focusing point.

In operation 730, the parameter operator 120 may confirm whether a position of the unnecessary focusing point extracted in operation 610 is identical to a position of the new unnecessary focusing point extracted in operation 720. When the position of the unnecessary focusing point extracted in operation 610 is identical to the position of the new unnecessary focusing point, the parameter operator 120 may perform operation 740. In addition, when the unnecessary focusing point extracted in operation 610 is different from the new unnecessary focusing point, the parameter operator 120 may perform operation 750.

In operation 740, the parameter operator 120 may readjust a final focusing parameter. Specifically, the parameter operator 120 may readjust a value of αₚ in proportion to the difference between the temperature of the unnecessary focusing point extracted in operation 610 and the temperature of the new unnecessary focusing point. In addition, the parameter operator 120 may readjust an unnecessary focusing point removal parameter according to the readjusted value of and may readjust the final focusing parameter using the readjusted unnecessary focusing point removal parameter. For example, the parameter operator 120 may further increase the value of αₚ as the difference between the temperature of the unnecessary focusing point extracted in operation 610 and the temperature of the new unnecessary focusing point increases.

In operation 750, the parameter operator 120 may determine a new unnecessary focusing point removal parameter for removing the new unnecessary focusing point.

In operation 760, the parameter operator 120 may determine a new final focusing parameter at which the unnecessary focusing point does not occur, by using the final focusing parameter and the new unnecessary focusing point removal parameter.

In operation 770, the electromagnetic wave generator 140 may generate electromagnetic waves corresponding to the final focusing parameter readjusted in operation 740 or the new final focusing parameter determined in operation 760. In addition, the electromagnetic wave irradiator 150 may irradiate the electromagnetic waves generated by the electromagnetic wave generator 140 to the object 101.

The components described in the embodiments may be implemented by hardware components including, for example, at least one digital signal processor (DSP), a processor, a controller, an application-specific integrated circuit (ASIC), a programmable logic element, such as a field programmable gate array (FPGA), other electronic devices, or combinations thereof. At least some of the functions or the processes described in the embodiments may be implemented by software, and the software may be recorded on a recording medium. The components, the functions, and the processes described in the embodiments may be implemented by a combination of hardware and software.

The method according to embodiments may be written in a computer-executable program and may be implemented as various recording media such as magnetic storage media, optical reading media, or digital storage media.

Various techniques described herein may be implemented in digital electronic circuitry, computer hardware, firmware, software, or combinations thereof. The techniques may be implemented as a computer program product, i.e., a computer program tangibly embodied in an information carrier, e.g., in a machine-readable storage device (for example, a computer-readable medium) or in a propagated signal, for processing by, or to control an operation of, a data processing apparatus, e.g., a programmable processor, a computer, or multiple computers. A computer program, such as the computer program(s) described above, may be written in any form of a programming language, including compiled or interpreted languages, and may be deployed in any form, including as a stand-alone program or as a module, a component, a subroutine, or other units suitable for use in a computing environment. A computer program may be deployed to be processed on one computer or multiple computers at one site or distributed across multiple sites and interconnected by a communication network.

Processors suitable for processing of a computer program include, by way of example, both general and special purpose microprocessors, and any one or more processors of any kind of digital computer. Generally, a processor will receive instructions and data from a read-only memory or a random-access memory, or both. Elements of a computer may include at least one processor for executing instructions and one or more memory devices for storing instructions and data. Generally, a computer also may include, or be operatively coupled to receive data from or transfer data to, or both, one or more mass storage devices for storing data, e.g., magnetic, magneto-optical disks, or optical disks. Examples of information carriers suitable for embodying computer program instructions and data include semiconductor memory devices, e.g., magnetic media such as hard disks, floppy disks, and magnetic tape, optical media such as compact disk read only memory (CD-ROM) or digital video disks (DVDs), magneto-optical media such as floptical disks, read-only memory (ROM), random-access memory (RAM), flash memory, erasable programmable ROM (EPROM), or electrically erasable programmable ROM (EEPROM). The processor and the memory may be supplemented by, or incorporated in special purpose logic circuitry.

In addition, non-transitory computer-readable media may be any available media that may be accessed by a computer and may include both computer storage media and transmission media.

Although the present specification includes details of a plurality of specific embodiments, the details should not be construed as limiting any invention or a scope that can be claimed, but rather should be construed as being descriptions of features that may be peculiar to specific embodiments of specific inventions. Specific features described in the present specification in the context of individual embodiments may be combined and implemented in a single embodiment. On the contrary, various features described in the context of a single embodiment may be implemented in a plurality of embodiments individually or in any appropriate sub-combination. Furthermore, although features may operate in a specific combination and may be initially depicted as being claimed, one or more features of a claimed combination may be excluded from the combination in some cases, and the claimed combination may be changed into a sub-combination or a modification of the sub-combination.

Likewise, although operations are depicted in a specific order in the drawings, it should not be understood that the operations must be performed in the depicted specific order or sequential order or all the shown operations must be performed in order to obtain a preferred result. In a specific case, multitasking and parallel processing may be advantageous. In addition, it should not be understood that the separation of various device components of the aforementioned embodiments is required for all the embodiments, and it should be understood that the aforementioned program components and apparatuses may be integrated into a single software product or packaged into multiple software products.

The embodiments disclosed in the present specification and the drawings are intended merely to present specific examples in order to aid in understanding of the present disclosure, but are not intended to limit the scope of the present disclosure. It will be apparent to those skilled in the art that various modifications based on the technical spirit of the present disclosure, as well as the disclosed embodiments, can be made.

What is claimed is:

1. A method of determining a focusing parameter of electromagnetic waves, the method comprising:
determining an initial focusing parameter based on a tomographic image of an object;
predicting a temperature change of the object, when assuming that electromagnetic waves are irradiated to the object according to the initial focusing parameter;
determining a final focusing parameter by optimizing a focusing parameter according to a prediction result of the temperature change; and
irradiating electromagnetic waves to the object according to the final focusing parameter,
wherein the prediction result of the temperature change indicates whether an unnecessary focusing point has occurred in the object.

2. The method of claim 1, wherein
the determining of the initial focusing parameter comprises:
converting the tomographic image into a numerical model including electromagnetic characteristics of a medium of the object;
determining an analysis result by performing an electromagnetic analysis on the numerical model; and
determining the initial focusing parameter based on the analysis result.

3. The method of claim 1, wherein
the predicting of the temperature change comprises:
when electromagnetic waves are irradiated to the object according to the initial focusing parameter, defining a focusing degree of electromagnetic waves as power loss density by predicting the focusing degree of electromagnetic waves for each area inside the object;
predicting the temperature change in each area inside the object, based on a temperature increase range for each area inside the object and the power loss density; and
confirming whether the unnecessary focusing point, which is an unintended energy focusing point of electromagnetic waves, has occurred by using a focusing target point corresponding to the temperature change in each area and the initial focusing parameter.

4. The method of claim 1, wherein
the determining of the final focusing parameter comprises, when the unnecessary focusing point is confirmed not to have occurred in the prediction result of the temperature change, determining the initial focusing parameter as the final focusing parameter.

5. The method of claim 1, wherein
the determining of the final focusing parameter comprises:
when the unnecessary focusing point is confirmed to have occurred in the prediction result of the temperature change, extracting the unnecessary focusing point;
determining an unnecessary focusing point removal parameter for removing the unnecessary focusing point; and
determining a new final focusing parameter at which the unnecessary focusing point does not occur, by using the initial focusing parameter and the unnecessary focusing point removal parameter.

6. The method of claim 5, wherein
the unnecessary focusing point removal parameter is a parameter corresponding to electromagnetic waves for generating, at the unnecessary focusing point, an electric field that has a same magnitude and an opposite phase with respect to an electric field generated at the unnecessary focusing point by the electromagnetic waves irradiated to the object according to the initial focusing parameter.

7. The method of claim 5, wherein
the determining of the final focusing parameter further comprises:
when electromagnetic waves are irradiated to the object according to the final focusing parameter, defining a focusing degree of electromagnetic waves as optimal power loss density by predicting the focusing degree of electromagnetic waves for each area inside the object;
predicting the temperature change in each area inside the object, based on a temperature increase range for each area inside the object and the optimal power loss density; and
confirming whether the unnecessary focusing point has occurred by using a focusing target point corresponding to the temperature change in each area and the final focusing parameter,
wherein the determining of the final focusing parameter is repeatedly performed until the unnecessary focusing point does not occur.

8. The method of claim 1, further comprising:
monitoring temperature of the object to which electromagnetic waves are irradiated according to the final focusing parameter; and
readjusting the final focusing parameter based on a monitoring result.

9. The method of claim 8, wherein
the readjusting of the final focusing parameter comprises:
confirming, based on the monitoring result, whether an unnecessary temperature increase has occurred, in which temperature increases above a threshold value in an area other than a focusing target point inside the object;
when the unnecessary temperature increase has occurred, extracting an area in which the unnecessary temperature increase has occurred as a new unnecessary focusing point;
when a position of the unnecessary focusing point is identical to a position of the new unnecessary focusing point, readjusting the final focusing parameter; and
irradiating electromagnetic waves to the object according to the readjusted final focusing parameter.

10. The method of claim 9, wherein
the readjusting of the final focusing parameter further comprises:
when the unnecessary focusing point is different from the new unnecessary focusing point, determining a new unnecessary focusing point removal parameter for removing the new unnecessary focusing point;
determining a new final focusing parameter at which the unnecessary focusing point does not occur, by using the final focusing parameter and the new unnecessary focusing point removal parameter; and
irradiating electromagnetic waves to the object according to the new final focusing parameter.

11. An electromagnetic wave energy treatment system comprising:
a parameter operator configured to determine an initial focusing parameter based on a tomographic image of an object, predict a temperature change of the object, when assuming that electromagnetic waves are irradiated to the object according to the initial focusing parameter, and determine a final focusing parameter by optimizing a focusing parameter according to a prediction result of the temperature change; and
an electromagnetic wave irradiator configured to irradiate electromagnetic waves to the object according to the final focusing parameter,
wherein the prediction result of the temperature change indicates whether an unnecessary focusing point has occurred in the object.

12. The electromagnetic wave energy treatment system of claim 11, wherein
the parameter operator is further configured to:
convert the tomographic image into a numerical model including electromagnetic characteristics of a medium of the object;
determine an analysis result by performing an electromagnetic analysis on the numerical model; and
determine the initial focusing parameter based on the analysis result.

13. The electromagnetic wave energy treatment system of claim 11, wherein
the parameter operator is further configured to:
when electromagnetic waves are irradiated to the object according to the initial focusing parameter, define a focusing degree of electromagnetic waves as power loss density by predicting the focusing degree of electromagnetic waves for each area inside the object;
predict the temperature change in each area inside the object, based on a temperature increase range for each area inside the object and the power loss density; and
confirm whether the unnecessary focusing point, which is an unintended energy focusing point of electromagnetic waves, has occurred by using a focusing target point corresponding to the temperature change in each area and the initial focusing parameter.

14. The electromagnetic wave energy treatment system of claim 11, wherein
the parameter operator is further configured to, when the unnecessary focusing point is confirmed not to have occurred in the prediction result of the temperature change, determine the initial focusing parameter as the final focusing parameter.

15. The electromagnetic wave energy treatment system of claim 11, wherein
the parameter operator is further configured to:
when the unnecessary focusing point is confirmed to have occurred in the prediction result of the temperature change, extract the unnecessary focusing point;
determine an unnecessary focusing point removal parameter for removing the unnecessary focusing point; and
determine a new final focusing parameter at which the unnecessary focusing point does not occur, by using the initial focusing parameter and the unnecessary focusing point removal parameter.

16. The electromagnetic wave energy treatment system of claim 15, wherein
the unnecessary focusing point removal parameter is a parameter corresponding to electromagnetic waves for generating, at the unnecessary focusing point, an electric field that has a same magnitude and an opposite phase with respect to an electric field generated at the unnecessary focusing point by the electromagnetic waves irradiated to the object according to the initial focusing parameter.

17. The electromagnetic wave energy treatment system of claim 15, wherein
the parameter operator is further configured to:
when electromagnetic waves are irradiated to the object according to the final focusing parameter, define a focusing degree of electromagnetic waves as optimal power loss density by predicting the focusing degree of electromagnetic waves for each area inside the object;
predict the temperature change in each area inside the object, based on a temperature increase range for each area inside the object and the optimal power loss density; and
confirm whether the unnecessary focusing point has occurred by using a focusing target point corresponding to the temperature change in each area and the final focusing parameter.

18. The electromagnetic wave energy treatment system of claim 11, further comprising:
a temperature monitor configured to monitor temperature of the object to which electromagnetic waves are irradiated according to the final focusing parameter,
wherein the parameter operator is further configured to readjust the final focusing parameter based on a monitoring result.

19. The electromagnetic wave energy treatment system of claim 18, wherein
the parameter operator is further configured to:
confirm, based on the monitoring result, whether an unnecessary temperature increase has occurred, in which temperature increases above a threshold value in an area other than a focusing target point inside the object;
when the unnecessary temperature increase has occurred, extract an area in which the unnecessary temperature increase has occurred as a new unnecessary focusing point; and
when a position of the unnecessary focusing point is identical to a position of the new unnecessary focusing point, readjust the final focusing parameter,
wherein the electromagnetic wave irradiator is further configured to irradiate electromagnetic waves to the object according to the readjusted final focusing parameter.

20. The electromagnetic wave energy treatment system of claim 19, wherein
the parameter operator is further configured to:
when the unnecessary focusing point is different from the new unnecessary focusing point, determine a new unnecessary focusing point removal parameter for removing the new unnecessary focusing point;
determine a new final focusing parameter at which the unnecessary focusing point does not occur, by using the final focusing parameter and the new unnecessary focusing point removal parameter,
wherein the electromagnetic wave irradiator is further configured to irradiate electromagnetic waves to the object according to the new final focusing parameter.
